(19) United States
(12) Patent Application Publication
(10) Pub. No.: US 2006/0100585 A1
(43) Pub. Date: May 11, 2006

(54) Safety device of butterfly needle

(76) Inventor: Tung-Hua Wang, Taipei County (TW)

Correspondence Address: Kang De Bao Biotechnology Co., Ltd., P.O. Box No. 6-57, Junghe, Taipei, 235, TW

(21) Appl. No.: 10/912,137

(22) Filed: Aug. 6, 2004

(51) Int. Cl. A61M 5/32; A61M 5/00

(52) U.S. Cl. 604/177; 604/192; 604/263

(57) ABSTRACT

A safety device of butterfly needle includes a sliding groove seat and a sliding groove cover. By assembling the sliding groove seat with the sliding groove cover, a case having sliding tracks at two sides thereof is formed. A butterfly needle having two wings can be accommodated in the case, and the two wings can be extended out from the sliding tracks at the two sides of the case. Thus, a user is still allowed to easily manipulate the butterfly needle by handholding the wings, and a needle head of the butterfly needle is freely extended and retracted within the case, such that the needle head is not randomly extended out of the case, thereby maintaining usage safety of the butterfly needle.

### BACKGROUND OF THE INVENTION

(a) Field of the Invention

The invention relates to a safety device of butterfly needle, and more particularly, to a safety device of butterfly needle, in that the safety device has an assembled case for handily accommodating a butterfly needle having a specific structure therein, while also being capable of maintaining satisfactory retracting and extending operations of the butterfly needle as well as providing original handiness of the butterfly needle, thereby achieving maximum practical values of the butterfly needle.

(b) Description of the Prior Art

A butterfly needle for medical use is a special injection needle having a structure different from that of a regular injection needle. A butterfly needle has two wings similar to those of a butterfly at a connecting portion between a needle body and a tube end. The wings are for facilitating grasp of a user in order to smoothly perform injection. The wings are also for favoring stabilization of the needle body when injection is completed. Although such type of butterfly needle has unique effects when put to application, it lacks appropriate safety measures due to the specific structure thereof and is only packaged with an ordinary packaging bag. When the butterfly needle is taken out from the bag, a user is frequently pricked by the needle and thus causing fears of the user. Also, safety measures again fall short for collection and recycling of the used needle to lead to perplexities of a handler. Hence, in order to elevate medical quality, it is vital that the prior butterfly needle be advanced to overcome the aforesaid usage inconveniences.

### SUMMARY OF THE INVENTION

The primary object of the invention is to provide a safety device of butterfly needle capable of overcoming the aforesaid drawbacks of prior butterfly needles, so as to offer a user with safety measures when using a butterfly needle while also maintaining original effects of the butterfly needle. To accomplish the primary object, the invention comprises a case assembled from a sliding groove seat and a sliding groove cover. The case has a sliding groove at an interior thereof, and sliding tracks at two sides thereof, so as to accommodate the butterfly needle in sliding groove and to extended two wings of the butterfly needle via the two side sliding tracks. Thus, a user can still manipulate the butterfly needle by handholding the two wings. With protection of the case, a needle head of the butterfly needle is prevented from pricking that may lead to infection or injuries of a user, and is also free from drawbacks of complicated processing after using the butterfly needle, thereby effectively overcoming usage issues and elevating practical values of the prior butterfly needle.

The secondary object of the invention is to provide a safety device of butterfly pin, wherein the sliding groove cover of the assembled case has locating protrusions at one end where the butterfly needle is extended out of the case, such that retaining effects are also provided when the butterfly needle is extended out of the case. Thus, the butterfly needle is remained at a stable state to maintain usage safety when being used for injection.

### BRIEF DESCRIPTION OF THE DRAWINGS

FIG. 1 shows an exploded elevational schematic view of an embodiment according to the invention.

FIG. 2 shows an elevational schematic view of an embodiment in assembly according to the invention.

FIG. 3 shows a butterfly needle being stored in an embodiment according to the invention.

FIG. 4 shows a butterfly needle being used for injection in an embodiment according to the invention.

### DETAILED DESCRIPTION OF THE PREFERRED EMBODIMENTS

To better understand structures and functions of the device according to the invention, detailed descriptions of a preferred embodiment shall be given with the accompanying drawings below.

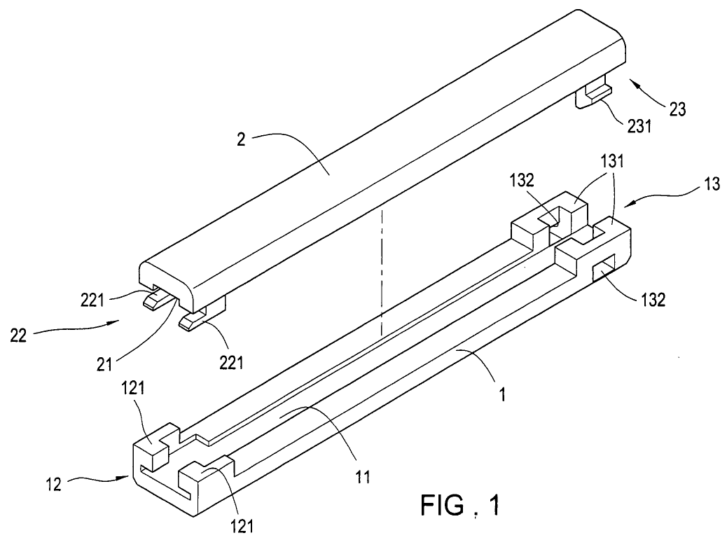

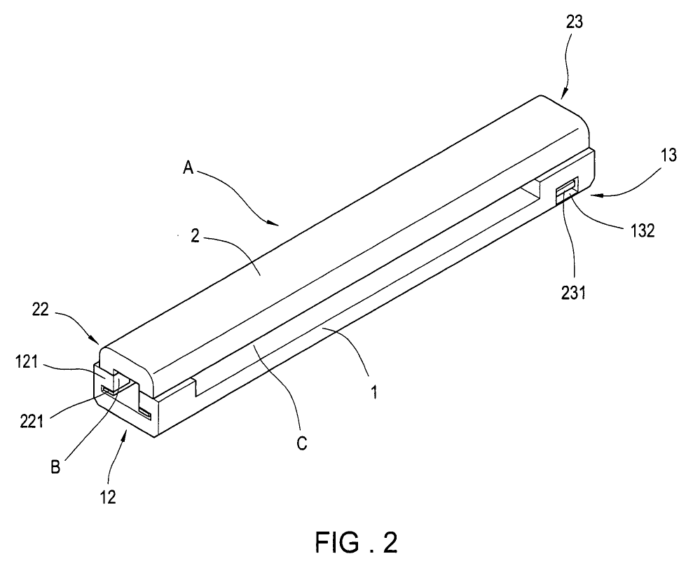

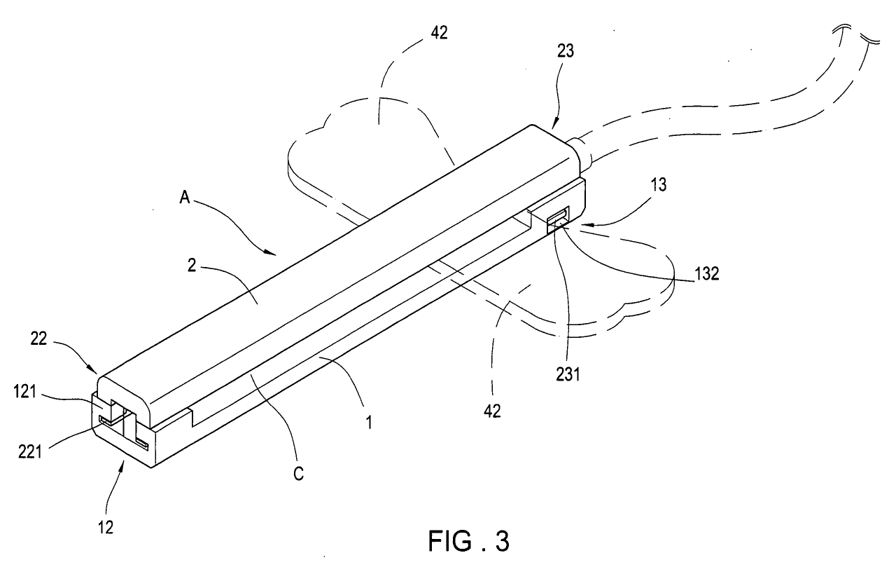

Referring to FIG. 1 showing an exploded elevational schematic view of an embodiment according to the invention, a device comprises a sliding groove seat 1 and a sliding groove cover 2. The sliding groove seat 1 has a long groove 11 at an upper surface thereof, and joining members 12 and 13 at two ends of the long groove 11. The sliding groove cover 2 is devised in a corresponding manner to the sliding groove seat 1; and has a long groove 21 at a lower surface thereof, and joining members 22 and 23. In this embodiment, each joining member 12 of the sliding groove seat 1 is disposed symmetrical L-shaped clasping plates 121 each having a lower portion thereof located above the long groove 11 with an appropriate distance in between. For corresponding with the clasping plates 121, the joining member 22 of the sliding groove cover 2 is symmetrically provided with two clasping hooks 221 at two sides of the long groove 21, thereby fastening the sliding groove seat 1 with the sliding groove cover 2 at one end. The joining member 13 at the other end of the sliding groove seat 1 is provided with arched clasping plates 13 at two sides of the long groove 11. Each clasping plate 131 has a fastening opening 132 at an inner wall thereof for corresponding with the joining member 23 at the other end of the sliding groove cover 2. The joining member 23 has insertion sections 231, which are extended from two sides of the long groove 21 and can be inserted into the arched clasping plates 131 at the sliding groove seat 1 to become fastened with the fastening openings 132 at the inner walls, thereby completing joining of the sliding groove seat 1 and the sliding groove cover 2 at the other end. Referring to FIG. 2, a case A is formed when the sliding groove seat 1 and the sliding groove cover 2 are joined. The case A has a sliding groove B formed by the two long grooves 11 and 12. Owing to the joining members 12, 13, 22 and 23 at two ends of the case A, the sliding groove B is provided with openings. Meanwhile, for that the joining members 12 and 13 at the two ends of the sliding groove seat 1 appear as projecting elements, sliding tracks C are developed at two sides of the case A, such that a butterfly needle 4 having a specific structure can be placed in the case A using the sliding groove B and the sliding tracks C. Referring to FIG. 3, a needle body 41 of the butterfly needle 4 is placed in the sliding groove B, and two side wings 42 thereof are extended out from the sliding tracks C at the two sides of the case A. Thus, a user is still allowed to handhold and to manipulate the wings 42 of the butterfly needle 4. When the butterfly needle 4 is located in the case A, safety measures are offered for being protected by the case A.

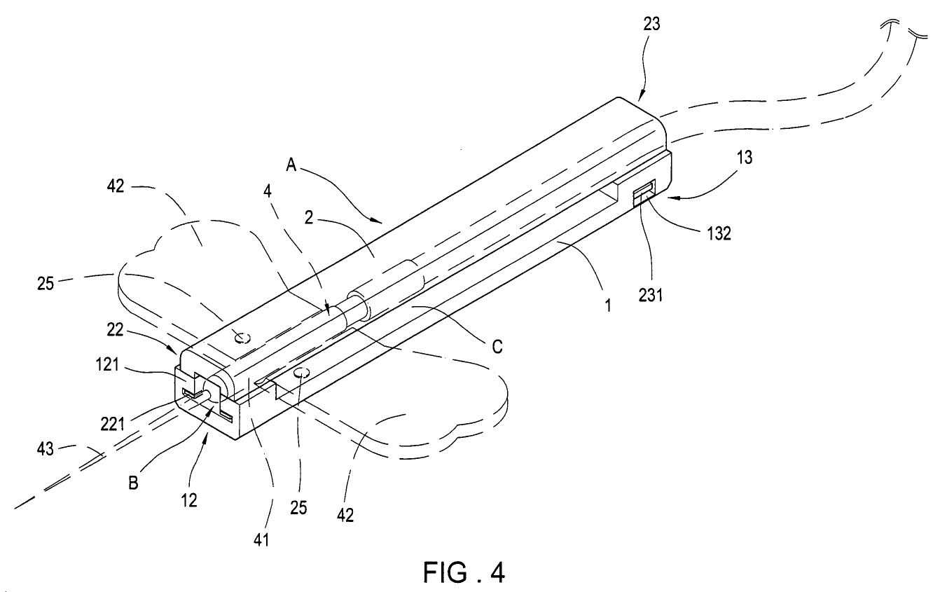

Furthermore, when using the butterfly needle 4, by applying slight forces to the wings 42, the butterfly needle 4 is the smoothly glided forward to extend a needle head 43 of the butterfly needle 4 out of the case A, thereby performing medical injection. Referring to FIG. 4, when the butterfly needle 4 is used for injection, stability required during injection is fulfilled since the case A is kept away from unwanted contact and is therefore remained unmoved. At a position where the butterfly needle 4 extends out of the case A, to similarly reduce the gap of the sliding groove C and to limit the position of the butterfly needle 4 within the case A, the sliding groove cover 2 is further provided with locating protrusions 25 at the sliding groove C, thereby reinforcing stability of the butterfly needle 4. When having completed injection, the butterfly needle 4 can be retracted into the case A to offer safety measures for collection and recycling of medical equipments. Therefore, the safety device according to the invention is indeed capable of effectively providing the butterfly needle 4 with usage safety effects and thus overcoming various drawbacks of prior butterfly needles.

Conclusive from the aforesaid descriptions, the safety device of butterfly needle according to the invention is capable of overcoming drawbacks of the prior butterfly needles. It is of course to be understood that the embodiment described herein is merely illustrative of the principles of the invention and that a wide variety of modifications thereto may be effected by persons skilled in the art without departing from the spirit and scope of the invention as set forth in the following claims.

What is claimed is:

1. A safety device of butterfly needle, comprising a sliding groove seat and a sliding groove cover; wherein:
the sliding groove seat has a long groove at an upper surface thereof, and projecting joining members at two ends of the long groove; and
the sliding groove seat and the sliding groove cover are joined by corresponding with each other to form a case having a sliding groove at an interior thereof and sliding tracks at two sides thereof.

2. The safety device of butterfly needle in accordance with claim 1, wherein the joining members are provided with symmetrical L-shaped clasping plates at two sides of the long groove thereof, and each L-shaped clasping plate has a lower portion thereof located above the long groove with a distance in between.

3. The safety device of butterfly needle in accordance with claim 1, wherein the joining members are provided with arched clasping plates at two sides of the long groove thereof, and each arched plate has a fastening opening at an inner wall thereof.

4. The safety device of butterfly needle in accordance with claim 1, wherein the joining members of the sliding groove cover have two symmetrical clasping hooks at two sides of the long groove.

5. The safety device of butterfly needle in accordance with claim 1, wherein the joining member of the sliding groove cover have insertion sections extended downward from two sides of the long groove thereof.

6. The safety device of butterfly needle in accordance with claim 1, wherein the case assembled from the sliding groove seat and the sliding groove cover, at a position where the butterfly needle is extended out of the case, is provided with protrusions for limiting a position of the butterfly needle within the case.
